(19) United States
(12) Patent Application Publication
(10) Pub. No.: US 2007/0142480 A1
(43) Pub. Date: Jun. 21, 2007

(54) Alleviation of pain in osteoarthritis by means of intra-articular implantation of perfluorodecalin.

(76) Inventor: Sarfaraz Niazi, Deerfield, IL (US)

Correspondence Address: TRAINER 1 LIMITED, KIMBERLEY ROAD, KIMBERLEY COURT, UNIT 26, LONDON, ENG, NW6-75L, GB

(21) Appl. No.: 11/306,155

(22) Filed: Dec. 18, 2005

(51) Int. Cl. A61K 31/025

(52) U.S. Cl. 514/747

(57) ABSTRACT

Methods of alleviating pain by the intra-articular application of perfluorodecalin are disclosed.

### FIELD OF INVENTION

This invention is related to the alleviation of pain in osteochondral lesions by means of intra-articular implantation of perfluorodecalin, a fully fluorinated fluorocarbon. The main objective of the invention is to introduce the new medical use of perfluorodecalin for the alleviation of pain when injected into joints wherein perfluorodecalin provides a lubrication and viscoelastic function that allows smooth movement of joints without appreciable pain.

### BACKGROUND OF THE INVENTION

Osteoarthritis (OA) is characterized by several pathological events, including a progressive erosion of the articular cartilage (particularly the weight bearing areas of the joint), synovial inflammation, which may contribute to disease progression, and changes in the lubricating properties of the synovial fluid (SF). Pharmacologic therapy for osteoarthritis is presently only palliative and is based on the use of analgesic or anti-inflammatory agents. Simple analgesics, however, do not provide enough of an effect to satisfy the needs of many OA patients, and anti-inflammatory drugs that are currently available do not have favorable risk-to-benefit ratio in typical patients with OA. Recently, a well known Cox-2 inhibitor, Vioxx® was recalled from the market for its deleterious effects on heart.

Controlled clinical trials, which include placebo groups, suggest that there is little to be gained over joint aspiration alone, or even over a simple needle prick. Glucocorticoids may however offer a small additional symptom benefit over one or two weeks. Intra-articular radiotherapy probably confers little benefit. Serious adverse effects are rare but local effects may occur in up to 10% of patients treated with viscosupplements.

A need remains, therefore, for therapies that will be analgesic, appropriately anti-inflammatory when necessary, and that may favorably alter the natural history of the disease. In knees with osteoarthritis, normal joint fluid, called synovial fluid, becomes thinner and loses its elasticity and viscosity. The diseased synovial fluid cannot provide “cushioning” in the knee joint. Without this cushioning, the cartilage in the knee joint may be more likely to wear down over time. This deterioration along with the loss of cushioning can contribute to pain and stiffness in the knee. Knee osteoarthritis is a common but often difficult problem to manage in primary care. Traditional non-surgical management, consisting of lifestyle modification, physical therapy and pharmacologic therapy (e.g., analgesics, anti-inflammatory medications), is often ineffective or leaves residual symptoms. Viscosupplementation is a newly available option for patients with symptomatic knee osteoarthritis that involves a series of intra-articular injections of hyaluronic acid, among other products.

Viscosupplementation is a newer medical concept that has as its therapeutic goal the restoration of rheological homeostasis in pathological structures such as osteoarthritic joints. When the normal viscoelasticity of a solid tissue compartment or the elastoviscosity of a liquid tissue compartment is decreased under pathological conditions, normal function and regenerative processes are impaired. By introducing viscosupplementation devices, the normal rheological state of such compartments is restored or augmented. These devices stay in the tissue compartment for various periods of time, depending on the nature of the viscosupplements and the pathophysiology of the tissue compartment. Viscosupplementation therapy is based on the concept of replenishing a normal physiological component of synovial fluid and cartilaginous tissue. Viscosupplementation therapy can restore the elastic and viscous properties of synovial fluid and thus recreate the intra-articular joint homeostasis that is disrupted in the degenerative joint. Clinical experience and studies of the hyaluronic acid products, hyaluronan and hylan G-F 20, seem to indicate beneficial effects with minimal adverse reactions in a significant number of patients. There are various products on the market for viscosupplementation; these include hyaluronan preparations of relatively low molecular weight (Hyalgan® and ARTZ®), a hyaluron preparation of intermediate molecular weight, but still lower molecular weight than that of the hyaluron in normal healthy synovial fluid (Orthovisc®), and a cross-linked hyaluron (a hylan) of high molecular weight (Synvisc®).

The exact mechanism of action of these products is unclear, although increasing the viscoelasticity of the synovial fluid appears to play a role. The exact indications for viscosupplementation are still evolving, but it currently can be considered for use in patients who have significant residual symptoms despite traditional non-pharmacologic and pharmacologic treatments. In addition, patients who are intolerant of traditional treatments (e.g., gastrointestinal problems related to anti-inflammatory medications) can be considered for these treatments.

It has been proven that the change of the intra-articular fluids produces a blockage of the nociceptors of subsynovial and capsular tissues and that, in addition to the mechanical factors of the osteochondral pathology, the fluids exert a relevant influence with their lubricating properties. Thus the change in viscosity of these fluids acts favorably on the painful osteochondral symptoms when sodium hyaluronate is instilled.

Present invention reveals another alternative in the management of pain in the knee or other body joints through the intra-articular application of perfluorodecalin based on the principles of viscoelastic properties of perfluorodecalin wherein implanting an artificial matrix that provides instant lubrication and expansion of local cavity relieves pain.

### SUMMARY OF THE INVENTION

This invention was developed to solve the problem related to the treatment of severe pain in osteoarthritis due to lack of lubrication. This invention introduces a method to achieve alleviation of pain through the intra-articular implantation of an artificial matrix in patients with grade I or II osteoarthritis in any joint of the human body. The product consists essentially of perfluorodecalin, sterilized by autoclaving and by injecting 0.5-2 mL in the target joint, with or without other analgesics or anesthetics and with or without other pharmaceutical adjuvants.

### DETAILED DESCRIPTION OF THE INVENTION

The product is applied by conventional intra-articular means with prior asepsis and antisepsis of the region. The preferred method for this invention's pain alleviation is the intra-articular application of perfluorodecalin either as pure liquid or in a suitable pharmaceutical dosage form such as a gel wherein the total amount of perfluorodecalin injected consists of a dose of approximately 1.5 mL of the formulation, when dealing with a large joint, or 0.50 mL for a small joint. The treatment can be applied repeatedly and periodically for an indefinite period of time without any side effects. On an average, the relief of pain lasts for at one week as perfluorodecalin is gradually removed from the injection site and eliminated from the body through the reticuloendothelial system (RES).

Perfluorodecalin is liquid fully fluorinated polycyclic compound which is chemically stable to acids and alkalis. It is also thermally stable up to 720° K. It is easily autoclaved at 121° C. It is insoluble in water and in traditional organic solvents; it is unlimited in mixing with other fluorocarbonic fluids. Approximately 45 mL of oxygen will dissolve in 100 mL of a perfluorocarbon liquid. Carbon dioxide is approximately 2.5 times more soluble than is oxygen. It is incombustible, non-explosive and non-toxic. It is available in three purified forms: CAS 306-94-5, which is a mixture of cisand transforms, the CAS 60433-11-6, which is cis form and CAS 60433-12-7, which is transform. The product used in this invention is a mixture of cis and trans form.

The safety of perfluorodecalin to humans is established as a perfluorodecalin formulation (emulsion) under the brand name of Fluosol® (Alpha Therapeutics) has been approved Under Section 505 of the Act Administered by the Center for Biologics Evaluation (N860909, Dec. 26, 1989) for direct intravenous administration to humans. Side effects to this formulation (Fluosol®) were observed in some patients due to complement activation caused by the Pluronic® surfactant used in Fluosol®. Several newer forms of dosage forms are available or under development utilizing perfluorocarbons for human administration for its oxygen carrying capacity. Examples include Oxygent™ (perfluoro-octyl bromide), Oxyflour® (Supercytes®), based on perfluorodichoroctane (C₈F₁₆C₁₂) with triglyceride and egg yolk lecithin. Acute single dose animal toxicity studies for these products have indicated a LD₅₀ of 55 g/Kg body weight.

Perfluorodeclain is readily removed from the body through reticuloendothelial system (RES) and has high in vivo stability, and produces no known pharmacologic response in humans or animals.

Perfluorodecalin is used widely in the field of ophthalmic surgery as a tool for maneuvering intraocular tissues and as a short- or medium-term vitreous substitute. The very high density of perfluorocarbons has made them of great interest to eye surgeons. The detachment of the retina from the back of the eye is a serious medical condition potentially leading to blindness. However, reattachment is now possible by passing perfluorocarbon into the eye. The heavy perfluorocarbon excludes the vitreous fluid from behind the retinal tear, gently pressing the retina back into place. The tear is sealed with a laser, and the perfluorocarbon is removed a few weeks later. In in vivo experiments, several types of lesions in retinal tissue have been described in conjunction with long-term perfluorodecalin treatment and these are attributed to its physical rather any pharmacologic toxic effects.

Perfluorodecalin is also used for intravenous administration for its potential utility as non-aqueous suspending vehicles for long-term in vivo delivery of therapeutic proteins.

Studies demonstrate the marked cytoprotectant effects of oxygenated perfluorodecalin and Pluronic F-68, both alone and/or in combination, for plant cells recovered from cryostorage. Such options offer alternative post-thaw handling strategies to cells of those plant species, which, normally, respond poorly to conventional recovery procedures.

The use of oxygen at high pressure to promote wound healing is well known. The nearer to the body's surface the less blood flow there is, and so oxygen supply is correspondingly reduced. Putting a patient into a high-pressure chamber increases the oxygen concentration at the skin's surface, accelerating healing. Oxygenated perfluorocarbons also increase surface oxygen concentration, but without the need for an expensive pressure chamber. They are especially useful for scars, leg ulcers and radiation burns. (J D Whitney, Heart and Lung, 1989, 18, 466. General information about high-pressure oxygen in wound healing.)

The utility of perfluorodecalin in preventing formation of adhesions and in providing viscosupplementation comes from its unique physical properties. Use of perfluorodecalin is described in the treatment of surgical adhesions (U.S. Pat. No. 6,235,796) by Niazi. Following is a comparison of physical properties of perfluorodecalin with water.

| | Density, | Vapor pressure at | Surface tension, | O₂ solubility, | CO₂ solubility, |
|---|---|---|---|---|---|
| | g/mL | 37° C., mm Hg | dynes/cm | mL/L | mL/L |
| Water | 1.0 | 47 | 72 | 30 | 570 |
| Perfluorodecalin | 1.95 | 14 | 15 | 490 | 1400 |

The low surface tension of perfluorodecalin provides for a mechanism that allows it to spread rapidly and evenly throughout the aqueous environment, leaving a fine film between the layers of tissues, reducing inflammation and speeding the healing process and thus reducing adhesion and attrition. The large capacity of perfluorodecalin to contain oxygen further assists in faster healing of wounds. Our clinical test supplies were provided by F2 Chemicals Ltd, (Lancashire, UK).

Perfluorodecalin is manufactured by passing a hydrocarbon over a heated bed of cobalt trifluoride. During this aggressive process some fragmentation and rearrangement does occur, leading to a variety of impurities. A comprehensive purification process eliminates virtually all unsaturated and hydrogen-containing components. What remains is a small number of other perfluorocarbons. These all have very similar boiling points, and other physical properties, to perfluorodecalin, and are likewise considered non-toxic. The clinical test product was non-pyrogenic and sterilized by autoclaving.

The main purpose of this invention is to create a new treatment for knee osteoarthritis that does not seem to be favorably responding to normal drug treatment and the patients may be required to undergo orthoscopic surgery. In knees with osteoarthritis, normal joint fluid, called synovial fluid, becomes thinner and loses its elasticity and viscosity. The diseased synovial fluid cannot provide “cushioning” in the knee joint. Without this cushioning, the cartilage in the knee joint may be more likely to wear down over time. This deterioration along with the loss of cushioning can contribute to pain and stiffness in the knee. Knee osteoarthritis is a common but often difficult problem to manage in primary care. Traditional non-surgical management, consisting of lifestyle modification, physical therapy and pharmacologic therapy (e.g., analgesics, anti-inflammatory medications), is often ineffective or leaves residual symptoms. Viscosupplementation is a newly available option for patients with symptomatic knee osteoarthritis that involves a series of intra-articular injections of hyaluronic acid, among other products.

There are various products on the market for viscosupplementation; these include hyaluronan preparations of relatively low molecular weight (Hyalgan® and ARTZ®), a hyaluronan preparation of intermediate molecular weight, but still lower molecular weight than that of the hyaluronan in normal healthy synovial fluid (Orthovisc®), and a cross-linked hyaluronan (a hylan) of high molecular weight (Synvisc®).

This invention discloses that administration of pefluorodecalin by intra-articular injection in a dose of about 0.75 to 2.25 mL once per week for per two weeks or longer, for as long as needed weeks yields similar effects as obtained by other products used for this purpose. Because of the fluid nature of the compound, the invention is also expected to act as a “shock absorber” to cushion the knee joint. There is also a need to develop a cheaper alternate to currently approved therapies by the US FDA. For example, Synvisc® therapy costs about $500 per knee for a series of three injections. Because of the nature of this product and the method of its manufacture, perfluorodecalin, can be made available to patients at a fraction of the cost currently incurred in such treatments. Also, the safety profile of perfluorodecalin is much more validated that any other compound currently used for this purpose.

The efficacy of treatment was determined by a validated clinical test method and side effects recorded. This was a single group, open-label study, including outpatients of both sexes, aged between 18 and 85 years, with symptomatic knee OA. All patients (25) underwent weekly intra-articular injections of perfluorodecalin for 5 consecutive weeks and were followed-up for 10 additional weeks. The safety and tolerability profile (primary end-point) was assessed by adverse event reporting. The secondary endpoint was efficacy evaluated by changes in the Western Ontario and McMaster Universities (WOMAC) score vs. baseline. Patient and physician satisfaction were also recorded. Intra-articular perfluorodecalin was generally well tolerated. The most frequent adverse event was pain at the injection site (10% of the injections); no serious treatment-related adverse events were reported. The WOMAC score was significantly reduced within the first 2 weeks of treatment (from 5±2 to 3±2;, p<0.001), further decreased by the end of the injection series (week 6: 2±1.5; p<0.001) and maintained during the follow-up. The WOMAC subscores were also significantly reduced from week 4 for ‘pain’ and from week 6 for ‘stiffness’ and ‘physical function’.

The functional result subsequent to the implantation of the product was very satisfactory for most of treated patients. The difference between the plain systemic drug management and the intra-articular application of perfluorodecalin is very evidently in favor of the latter. It must be considered that the plain intra-articular rheological change (viscosity, elasticity and plasticity) reduces the pain and stimulates a synovial response, changing the viscoelastic features of the fluid.

Conclusions

In the present study, intra-articular perfluorodecalin was well tolerated and safe in patients with symptomatic knee OA. Based on the sustained improvements in WOMAC score and subscores, a carry-over effect lasting for at least 3 weeks after the last injection are proposed. These results further confirm the evidence of efficacy and safety of intra-articular perfluorodecalin in the management of knee OA. The treatment of pain in osteochondral lesions using implantation with perfluorodecalin has proven to have a significantly favorable clinical response compared with the conventional treatment, particularly in those patients who have turned refractory to analgesics. The experts in the technique will recognize that the preferred modes may be altered or amended without straying away from the true spirit and scope of the invention as defined in the enclosed claims.

### PUBLICATIONS ON VISCOSUPPLEMENTATION

1: Theiler R, Bruhimann P. Overall tolerability and analgesic activity of intra-articular sodium hyaluronate in the treatment of knee osteoarthritis. Curr Med Res Opin. 2005 November; 21(11): 1727-33.

2: Cunha P L, Castro R R, Rocha F A, de Paula R C, Feitosa J P. Low viscosity hydrogel of guar gum: preparation and physicochemical characterization. Int J Biol Macromol. 2005 Oct. 30; 37(1-2): 99-104.

3: Migliore A, Tormenta S, Martin Martin L S, lannessi F, Massafra U, Carloni E, Monno D, Alimonti A, Granata M. The symptomatic effects of intra-articular administration of hylan G-F 20 on osteoarthritis of the hip: clinical data of 6 months follow-up. Clin Rheumatol matol. 2005 Oct. 25: 1-5

4: Salk R, Chang T, D'Costa W, Soomekh D, Grogan K. Viscosupplementation (hyaluronans) in the treatment of ankle osteoarthritis. Clin Podiatr Med Surg North Am. 2005 October; 22(4): 585-97, vii.

5: Conrozier T, Vignon E. Is there evidence to support the inclusion of viscosupplementation in the treatment paradigm for patients with hip osteoarthritis? Clin Exp Rheumatol. 2005 September-October; 23(5): 711-6.

6: Modawal A, Ferrer M, Choi H K, Castle J A. Hyaluronic acid injections relieve knee pain. J Fam Pract. 2005 September; 54(9): 758-67.

7: Xinmin Y, Jian H. Treatment of temporomandibular joint osteoarthritis with viscosupplementation and arthrocentesis on rabbit model. Oral Surg Oral Med Oral Pathol Oral Radiol Endod. 2005 September; 100(3): e35-8

8: Tytherleigh-Strong G, Hurtig M, Miniaci A. Intra-articular hyaluronan following autogenous osteochondral grafting of the knee. Arthroscopy. 2005 August; 21(8): 999-1005.

9: Albert C, Brocq O, Gerard D, Roux C, Euller-Ziegler L. Septic knee arthritis after intra-articular hyaluronate injection Two case reports. Joint Bone Spine. 2005 Jul. 18;

10: Kotevoglu N, lyibozkurt P C, Hiz O, Toktas H, Kuran B. A prospective randomised controlled clinical trial comparing the efficacy of different molecular weight hyaluronan solutions in the treatment of knee osteoarthritis. Rheumatol Int. 2005 Jun. 15;

11: Tikiz C, Unlu Z, Sener A, Efe M, Tuzun C. Comparison of the efficacy of lower and higher molecular weight viscosupplementation in the treatment of hip osteoarthritis. Clin Rheumatol. 2005 June; 24(3): 244-50.

12: Migliore A, Tormenta S, Martin L S, Valente C, Massafra U, Granata M, Alimonti A. Open pilot study of ultrasound-guided intra-articular injection of hylan G-F 20 (Synvisc) in the treatment of symptomatic hip osteoarthritis. Clin Rheumatol. 2005 June; 24(3): 285-9.

13: Blumstein H, Gorevic P D. Rheumatologic illnesses: treatment strategies for older adults. Geriatrics. 2005 June; 60(6): 28-35.

14: Caglar-Yagci H, Unsal S, Yagci I, Dulgeroglu D, Ozel S. Safety and efficacy of ultrasound-guided intra-articular hylan G-F 20 injection in osteoarthritis of the hip: a pilot study. Rheumatol Int. 2005 June; 25(5): 341-4.

15: Tehranzadeh J, Booya F, Root J. Cartilage metabolism in osteoarthritis and the influence of viscosupplementation and steroid: a review. Acta Radiol. 2005 May; 46(3): 288-96.

16: Jones K B, Patel P P, DeYoung B R, Buckwalter J A. Viscosupplementation pseudo-tumor. A case report. J Bone Joint Surg Am. 2005 May; 87(5): 1113-9.

17: Bellamy N, Campbell J, Robinson V, Gee T, Bourne R, Wells G. Viscosupplementation for the treatment of osteoarthritis of the knee. Cochrane Database Syst Rev. 2005 Apr. 18; (2): CD005321.

18: Snibbe J C, Gambardella R A. Treatment options for osteoarthritis. Orthopedics. 2005 February; 28(2 Suppl): s215-20.

19: Shikhman A R, Amiel D, D'Lima D, Hwang S B, Hu C, Xu A, Hashimoto S, Kobayashi K, Sasho T, Lotz M K. Chondroprotective activity of N-acetylglucosamine in rabbits with experimental osteoarthritis. Ann Rheum Dis. 2005 January; 64(1): 89-94.

20: Snibbe J C, Gambardella R A. Use of injections for osteoarthritis in joints and sports activity. Clin Sports Med. 2005 January; 24(1): 83-91.

21: Fajardo M, Di Cesare P E. Disease-modifying therapies for osteoarthritis: current status. Drugs Aging. 2005; 22(2): 141-61.

22: Clarke S, Lock V, Duddy J, Sharif M, Newman J H, Kirwan J R. Intra-articular hylan G-F 20 (Synvisc) in the management of patellofemoral osteoarthritis of the knee (POAK). Knee. 2005 January; 12(1): 57-62.

23: Migliore A, Tormenta S, Valente C, Massafra U, Martin Martin L S, Carmenini E, Bernardini A, Alimonti A. Intra-articular treatment with Hylan G-F 20 under ultrasound guidance in hip osteoarthritis. Clinical results after 12 months follow-up. Reumatismo. 2005 January-March; 57(1): 36-43.

24: Magilavy D B, McPherson J M, Polisson R. Pseudoseptic reactions to Hylan viscosupplementation: diagnosis and treatment. Clin Orthop Relat Res. 2004 December; (429): 349-50; author reply 350-1.

25: Campbell D G, Angel K R, Dobson P J, Lewis P L, Tandon S. Experiences of viscosupplementation for knee osteoarthritis. Aust Fam Physician. 2004 October; 33(10): 863-4.

26: Wind W M Jr, Smolinski R J. Reliability of common knee injection sites with low-volume injections. J Arthroplasty. 2004 October; 19(7): 858-61.

27: Buchard P A. latrogenic rheumatic diseases. Rev Med Suisse Romande. 2004 September; 124(9): 551-5.

28: Fukuda K. Intra-articular injection of hyaluronan for the treatment of knee osteoarthritis. Clin Calcium. 2004 July; 14(7): 103-7.

29: Jay G D. Current thinking on viscosupplementation in osteoarthritis. Med Health R I. 2004 July; 87(7): 213-5.

30: Coutts R D, Waddell D D. Viscosupplementation for osteoarthritis of the knee. Orthopedics. 2004 May; 27(5): 470-1.

31: Lee S, Park D, Chmell S J. Viscosupplementation with hylan G-F 20 (Synvisc): pain and mobility observations from 74 consecutive patients. J Knee Surg. 2004 April; 17(2): 73-7.

32: Aggarwal A, Sempowski I P. Hyaluronic acid injections for knee osteoarthritis. Systematic review of the literature. Can Fam Physician. 2004 February; 50: 249-56.

33: Goldberg V M, Coutts R D. Pseudoseptic reactions to hylan viscosupplementation: diagnosis and treatment. Clin Orthop Relat Res. 2004 February; (419): 130-7.

34: Caborn D, Rush J, Lanzer W, Parenti D, Murray C; Synvisc 901 Study Group. A randomized, single-blind comparison of the efficacy and tolerability of hylan G-F 20 and triamcinolone hexacetonide in patients with osteoarthritis of the knee. J Rheumatol. 2004 February; 31(2): 333-43.

35: Zhong W Q, Zhou G. lntraarticular injection-dilatation and later lavage plus viscosupplementation of TMJ for the treatment of anterior disc displacement without reduction. Shanghai Kou Qiang Yi Xue. 2004 February; 13(1): 23-6.

36: Balazs E A. Viscosupplementation for treatment of osteoarthritis: from initial discovery to current status and results. Surg Technol Int. 2004; 12: 278-89.

37: Zardawi I M. Granulomatous inflammation after Hylan G-F 20 viscosupplementation of the knee. J Bone Joint Surg Am. 2003 December; 85-A(12): 2484; author reply 2484-5.

38: Marino AA, Dunn S, Waddell D D. Granulomatous inflammation after Hylan G-F 20 viscosupplementation of the knee. J Bone Joint Surg Am. 2003 October; 85-A(10): 2051-2; author reply 2052-3.

39: Conrozier T, Bertin P, Mathieu P, Chariot J, Bailleul F, Treves R, Vignon E, Chevalier X. Intra-articular injections of hylan G-F 20 in patients with symptomatic hip osteoarthritis: an open-label, multicentre, pilot study. Clin Exp Rheumatol. 2003 September-October; 21(5): 605-10.

40: Kahan A, Lieu P L, Salin L. Prospective randomized study comparing the medicoeconomic benefits of Hylan GF-20 vs. conventional treatment in knee osteoarthritis. Joint Bone Spine. 2003 August; 70(4): 276-81.

41: Marshall K W. Intra-articular hyaluronan therapy. Foot Ankle Clin. 2003 June; 8(2): 221-32, viii.

42: Cardone D A, Tallia A F. Diagnostic and therapeutic injection of the hip and knee. Am Fam Physician. 2003 May 15; 67(10): 2147-52.

43: Noain E, Sancez-Villares J J, Lasanta P J, Gonzalez Arteaga F J. Acute local reaction to intra-articular infiltration with synvisc (Hylan GF20). About two cases. An Sist Sanit Navar. 2003 May-August; 26(2): 283-5.

44: Vad V B, Bhat A L, Sculco T P, Wickiewicz T L. Management of knee osteoarthritis: knee lavage combined with hylan versus hylan alone. Arch Phys Med Rehabil. 2003 May; 84(5): 634-7.

45: Mont M A, Etienne G. Sequelae of Hylan G-F 20 viscosupplementation of the knee. J Bone Joint Surg Am. 2003 May; 85-A(5): 967-8; author reply 968-9.

46: Migliore A, Martin L S, Alimonti A, Valente C, Tormenta S. Efficacy and safety of viscosupplementation by ultrasound-guided intra-articular injection in osteoarthritis of the hip. Osteoarthritis Cartilage. 2003 April; 11 (4): 305-6.

47: Williams J M, Rayan V, Sumner D R, Thonar E J. The use of intra-articular Nahyaluronate as a potential chondroprotective device in experimentally induced acute articular cartilage injury and repair in rabbits. J Orthop Res. 2003 March; 21(2): 305-11.

48: Conrozier T, Mathieu P, Schott A M, Laurent I, Hajri T, Crozes P, Grand P, Laurent H, Marchand F, Meignan F, Noel E, Rozand Y, Savoye J F, Vignon E. Factors predicting long-term efficacy of Hylan GF-20 viscosupplementation in knee osteoarthritis. Joint Bone Spine. 2003 March; 70(2): 128-33.

49: Conrozier T, Flipo R M. Current management of osteoarthritis. Part 1: pharmacological strategies. Rev Med Interne. 2003 March; 24(3): 183-8.

50: Balazs E A. Analgesic effect of elastoviscous hyaluronan solutions and the treatment of arthritic pain. Cells Tissues Organs. 2003; 174(1-2): 49-62.

51: Waddell D D. The tolerability of viscosupplementation: low incidence and clinical management of local adverse events. Curr Med Res Opin. 2003; 19(7): 575-80.

52: Espallargues M, Pons J M. Efficacy and safety of viscosupplementation with Hylan G-F 20 for the treatment of knee osteoarthritis: a systematic review. Int J Technol Assess Health Care. 2003 Winter; 19(1): 41-56.

53: Toh E M, Prasad P S, Teanby D. Correlating the efficacy of knee viscosupplementation with osteoarthritic changes on roentgenological examination. Knee. 2002 December; 9(4): 321-30.

54: Pandolfi S, Exer P, Schwarz H A. Viscosupplementation in arthrosis. Ther Umsch. 2002 October; 59(10): 545-9.

55: Tadmor R, Chen N, Israelachvili J N. Thin film rheology and lubricity of hyaluronic acid solutions at a normal physiological concentration. J Biomed Mater Res. 2002 Sep. 15; 61(4): 514-23.

56: Pleimann J H, Davis W H, Cohen B E, Anderson R B. Viscosupplementation for the arthritic ankle. Foot Ankle Clin. 2002 September; 7(3): 489-94.

57: Geier K A, Keeperman J B, Sproul R C, Roth K, Reynolds H M. Viscosupplementation: a new treatment option for osteoarthritis. Orthop Nurs. 2002 September-October; 21(5): 25-32; quiz 32-4.

58: Chen A L, Desai P, Adler E M, Di Cesare P E. Granulomatous inflammation after Hylan G-F 20 viscosupplementation of the knee: a report of six cases. J Bone Joint Surg Am. 2002 July; 84-A(7): 1142-7.

59: Torrance G W, Raynauld J P, Walker V, Goldsmith C H, Bellamy N, Band P A, Schultz M, Tugwell P; Canadian Knee OA Study Group. A prospective, randomized, pragmatic, health outcomes trial evaluating the incorporation of hylan G-F 20 into the treatment paradigm for patients with knee osteoarthritis (Part 2 of 2): economic results. Osteoarthritis Cartilage. 2002 July; 10(7): 518-27.

60: Raynauld J P, Torrance G W, Band P A, Goldsmith C H, Tugwell P, Walker V, Schultz M, Bellamy N; Canadian Knee OA Study Group. A prospective, randomized, pragmatic health outcomes trial evaluating the incorporation of hylan G-F 20 into the treatment paradigm for patients with knee osteoarthritis (Part 1 of 2): clinical results. Osteoarthritis Cartilage. 2002 July; 10(7): 506-17.

61: Brocq O, Tran G, Breuil V, Grisot C, Flory P, Euller-Ziegler L. Hip osteoarthritis: short-term efficacy and safety of viscosupplementation by hylan G-F 20. An open-label study in 22 patients. Joint Bone Spine. 2002 June; 69(4): 388-91.

62: Nozaki H. Injection technique of intra-articular hyaluronic acid for knee osteoarthritis and periarthritis of shoulder. Clin Calcium. 2002 January; 12(1): 98-103.

63: Vad V, Hong H M, Zazzali M, Agi N, Basrai D. Exercise recommendations in athletes with early osteoarthritis of the knee. Sports Med. 2002; 32(11): 729-39.

64: Rees J D, Wojtulewski J A. Systemic reaction to viscosupplementation for knee osteoarthritis. Rheumatology (Oxford). 2001 December; 40(12): 1425-6.

65: Luo Y, Prestwich G D. Hyaluronic acid-N-hydroxysuccinimide: a useful intermediate for bioconjugation. Bioconjug Chem. 2001 November-December; 12(6): 1085-8.

66: Milas M, Rinaudo M, Roure I, Al-Assaf S, Phillips G O, Williams P A. Comparative rheological behavior of hyaluronan from bacterial and animal sources with cross-linked hyaluronan (hylan) in aqueous solution. Biopolymers. 2001 Oct. 5; 59(4): 191-204.

67: Waddell D, Rein A, Panarites C, Coleman P M, Weiss C. Cost implications of introducing an alternative treatment for patients with osteoarthritis of the knee in a managed care setti ng. Am J Manag Care. 2001 October; 7(10): 981-91.

68: McCarberg B H, Herr K A; American Academy of Pain Medicine. Osteoarthritis. How to manage pain and improve patient function. Geriatrics. 2001 October; 56(10): 14-7, 20-2, 24.

69: Vertullo C. Management of the osteoarthritic knee. New advances in nonoperative therapy. Aust Fam Physician. 2001 September; 30(9): 853-7.

70: Kirwan J. Is there a place for intra-articular hyaluronate in osteoarthritis of the knee? Knee. 2001 June; 8(2): 93-101.

71: Miller E H. Viscosupplementation: therapeutic mechanisms and clinical potential in osteoarthritis of the knee. J Am Acad Orthop Surg. 2001 March-April; 9(2): 146-7.

72: Pelletier S, Hubert P, Payan E, Marchal P, Choplin L, Dellacherie E. Amphiphilic derivatives of sodium alginate and hyaluronate for cartilage repair: rheological properties. J Biomed Mater Res. 2001 January; 54(1): 102-8.

73: Marshall K W. Intra-articular hyaluronan therapy. Curr Opin Rheumatol. 2000 September; 12(5): 468-74.

74: Watterson J R, Esdaile J M. Viscosupplementation: therapeutic mechanisms and clinical potential in osteoarthritis of the knee. J Am Acad Orthop Surg. 2000 September-October; 8(5): 277-84.

75: Wen D Y. Intra-articular hyaluronic acid injections for knee osteoarthritis. Am Fam Physician. 2000 Aug. 1; 62(3): 565-70, 572.

76: Adams M E, Lussier A J, Peyron J G. A risk-benefit assessment of injections of hyaluronan and its derivatives in the treatment of osteoarthritis of the knee. Drug Saf. 2000 August; 23(2): 115-30.

77: Allard S, O'Regan M. The role of elastoviscosity in the efficacy of viscosupplementation for osteoarthritis of the knee: a comparison of hylan G-F 20 and a lower-molecular-weight hyaluronan. Clin Ther. 2000 June; 22(6): 792-5.

78: Payne M W, Petrella R J. Viscosupplementation effect on proprioception in the osteoarthritic knee. Arch Phys Med Rehabil. 2000 May; 81(5): 598-603.

79: Marshall K W, Manolopoulos V, Mancer K, Staples J, Damyanovich A. Amelioration of disease severity by intraarticular hylan therapy in bilateral canine osteoarthritis. J Orthop Res. 2000 May; 18(3): 416-25.

80: Waddell D D. Viscosupplementation treatments. J South Orthop Assoc. 2000 Spring; 9(1): 79.

81: Wright K E, Maurer S G, Di Cesare P E. Viscosupplementation for osteoarthritis. Am J Orthop. 2000 February; 29(2): 80-8; discussion 88-9.

82: Calvillo O, Skaribas I, Turnipseed J. Anatomy and pathophysiology of the sacroiliac joint. Curr Rev Pain. 2000; 4(5): 356-61.

83: Bellamy N, Goldstein L D, Tekanoff R A; Support, Non-U.S. Gov't. Continuing medical education-driven skills acquisition and impact on improved patient outcomes in family practice setting. J Contin Educ Health Prof. 2000 Winter; 20(1): 52-61.

84: Cefalu C A, Waddell D S. Viscosupplementation: treatment alternative for osteoarthritis of the knee. Geriatrics. 1999 October; 54(10): 51-4, 57.

85: Wobig M, Bach G, Beks P, Dickhut A, Runzheimer J, Schwieger G, Vetter G, Balazs E. The role of elastoviscosity in the efficacy of viscosupplementation for osteoarthritis of the knee: a comparison of hylan G-F 20 and a lower-molecular-weight hyaluronan. Clin Ther. 1999 September; 21(9): 1549-62.

86: Uebelhart D, Williams J M. Effects of hyaluronic acid on cartilage degradation. Curr Opin Rheumatol. 1999 September; 11 (5): 427-35.

87: Simon L S. Viscosupplementation therapy with intra-articular hyaluronic acid. Fact or fantasy? Rheum Dis Clin North Am. 1999 May; 25(2): 345-57.

88: Srejic U, Calvillo O, Kabakibou K. Viscosupplementation: a new concept in the treatment of sacroiliac joint syndrome: a preliminary report of four cases. Reg Anesth Pain Med. 1999January-February; 24(1): 84-8.

89: Cohen M D. Hyaluronic acid treatment (viscosupplementation) for OA of the knee. Bull Rheum Dis. 1998 November; 47(7): 4-7.

90: Marshall K W. Viscosupplementation for osteoarthritis: current status, unresolved issues, and future directions. J Rheumatol. 1998 November; 25(11): 2056-8.

91: Jay G D, Haberstroh K, Cha C J. Comparison of the boundary-lubricating ability of bovine synovial fluid, lubricin, and Healon. J Biomed Mater Res. 1998 Jun. 5; 40(3): 414-8.

92: Wobig M, Dickhut A, Maier R, Vetter G. Viscosupplementation with hylan G-F 20: a 26-week controlled trial of efficacy and safety in the osteoarthritic knee. Clin Ther. 1998 May-June; 20(3): 410-23.

93: Vercruysse K P, Prestwich G D. Hyaluronate derivatives in drug delivery. Crit Rev Ther Drug Carrier Syst. 1998; 15(5): 513-55.

94: Kirwan J R, Rankin E. Intra-articular therapy in osteoarthritis. Baillieres Clin Rheumatol. 1997 November; 11 (4): 769-94.

95: Lussier A, Cividino A A, McFarlane C A, Olszynski W P, Potashner W J, De Medicis R. Viscosupplementation with hylan for the treatment of osteoarthritis: findings from clinical practice in Canada. J Rheumatol. 1996 September; 23(9): 1579-85.

96: al-Assaf S, Meadows J, Phillips G O, Williams P A. The application of shear and extensional viscosity measurements to assess the potential of hylan in viscosupplementation. Biorheology. 1996 July-October; 33(4-5): 319-32.

97: Adams M E, Atkinson M H, Lussier A J, Schulz J L, Siminovitch K A, Wade J P, Zummer M. The role of viscosupplementation with hylan G-F 20 (Synvisc) in the treatment of osteoarthritis of the knee: a Canadian multicenter trial comparing hylan G-F 20 alone, hylan G-F 20 with non-steroidal anti-inflammatory drugs (NSAIDs) and NSAIDs alone. Osteoarthritis Cartilage. 1995 December; 3(4): 213-25.

98: Yustin D, Kryshtalskyj B, Galea A. Use of Hylan G-F 20 for viscosupplementation of the temporomandibular joint for the management of osteoarthritis: a case report. J Orofac Pain. 1995 Fall; 9(4): 375-9.

99: Xiang Y, Shi G, Yuan G. Hyaluronic acid viscosupplementation in the treatment of osteoarthritis. Zhonghua Nei Ke Za Zhi. 1995 January; 34(1): 58-60.

100: Balazs E A, Denlinger J L. Viscosupplementation: a new concept in the treatment of osteoarthritis. J Rheumatol Suppl. 1993 August; 39: 3-9.

101: Pelletier J P, Martel-Pelletier J. The pathophysiology of osteoarthritis and the implication of the use of hyaluronan and hylan as therapeutic agents in viscosupplementation. J Rheumatol Suppl. 1993 August; 39: 19-24.

102: Adams M E. An analysis of clinical studies of the use of crosslinked hyaluronan, hylan, in the treatment of osteoarthritis. J Rheumatol Suppl. 1993 August; 39: 16-8.

103: Peyron J G. Intra-articular hyaluronan injections in the treatment of osteoarthritis state-of-the-art review. J Rheumatol Suppl. 1993 August; 39: 10-5.

104: Peyron J G. A new approach to the treatment of osteoarthritis: viscosupplementation. Osteoarthritis Cartilage. 1993 April; 1(2): 85-7.

105: Balazs E A, Denlinger J L. Clinical uses of hyaluronan. Ciba Found Symp. 1989; 143: 265-75; discussion 275-80, 281-5.

### REVIEW ARTICLES ON PERFLUORODECALIN

1: Lowe K C. Engineering blood: synthetic substitutes from fluorinated compounds. Tissue Eng. 2003 June; 9(3): 389-99.

2: Lowe K C. Second-generation perfluorocarbon emulsion blood substitutes. Artif Cells Blood Substit Immobil Biotechnol. 2000January; 28(1): 25-38.

3: Spence R K, Norcross E D, Costabile J, McCoy S, Cernaianu A C, Alexander J B, Pello M J, Atabek U, Camishion R C. Perfluorocarbons as blood substitutes: the early years. Experience with FluosoIDA-20% in the 1980s. Artif Cells Blood Substit Immobil Biotechnol. 1994; 22(4): 955-63.

4: Korobelnik J F, Nabet L, Frau E, Elmaleh C, Hanna K, Pouliquen Y. Use of perfluorocarbon solutions in the surgical treatment of posteriorluxation of the lens. J Fr Ophtalmol. 1992; 15(4): 235-42.

5: Labrude P. Current research on oxygen carriers for transfusion: hemoglobin solutions and fluorocarbon emulsions. Ann Pharm Fr. 1992; 50(5-6): 250-66.

6: Larochkin V S, Koziner V B. Problem of creating “artificial blood” based on organofluorine compounds. Patol Fiziol Eksp Ter. 1981 May-June; (3): 78-87.

What is claimed is:

1. A therapeutic method for the alleviation of pain caused by osteoarthritis, comprising the intra-articular implantation of perfluorodecalin in a pharmaceutically elegant dosage form.

2. The therapeutic method according to claim 1 for a joint selected from the group consisting of: knee, shoulder and sacroiliac.

3. The therapeutic method according to claim 1 for a joint selected from the group consisting of coxofemoral, ankle and elbow.

4. The therapeutic method according to claim 1 for a joint selected from the group consisting of interphalangeal and wrist.

5. The therapeutic method according to claim 1, wherein perfluorodecalin is combined with other pain relievers.

6. The therapeutic method according to claim 1, wherein perfluorodecalin is combined with anesthetic agents.

7. The therapeutic method according go claim 1, wherein perfluorodecalin is oxygenated prior to implantation.
